(19) United States
(12) Patent Application Publication
(10) Pub. No.: US 2009/0105236 A1
(43) Pub. Date: Apr. 23, 2009

(54) NEW PHARMACEUTICALLY-ACTIVE COMPOUNDS FOR THE TREATMENT OF RESPIRATORY DISEASES

(75) Inventors: Ingo KONETZKI, Warthausen (DE); Thierry BOUYSSOU, Warthausen (DE); Philipp LUSTENBERGER, Basel (CH); Andreas SCHNAPP, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim (DE)

(21) Appl. No.: 12/338,333

(22) Filed: Dec. 18, 2008

Related U.S. Application Data
(63) Continuation of application No. 11/276,307, filed on Feb. 23, 2006, now abandoned.

(30) Foreign Application Priority Data
Feb. 24, 2005 (DE) 10 2005 008921.6

(51) Int. Cl. A61K 31/536; C07D 265/14; A61P 11/00

(52) U.S. Cl. 514/229.8; 514/230.5; 544/92; 544/71

(57) ABSTRACT

The present invention relates to the use of the compounds of general formula 1

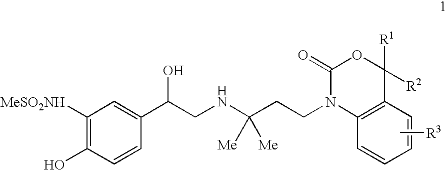

wherein the groups R¹, R² and R³ may have the meanings specified in the claims and in the description, for preparing a pharmaceutical composition for the treatment of respiratory complaints, as well as new compounds of formula 1, processes for preparing them, and pharmaceutical formulations containing them.

### RELATED APPLICATION

This application is a continuation of U.S. application Ser. No. 11/276,307 filed Feb. 23, 2006, which claims priority to German Application No. 10 2005 008921.6, filed Feb. 24, 2005, the contents of which are incorporated by reference in their entireties.

### FIELD OF THE INVENTION

The present invention relates to the use of the compounds of general formula 1

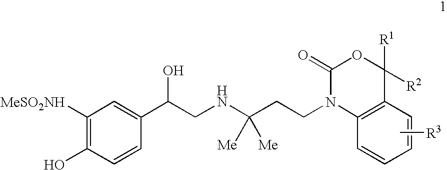

wherein the groups R¹, R² and R³ may have the meanings specified in the claims and in the description, for preparing a pharmaceutical composition for the treatment of respiratory complaints, as well as new compounds of formula 1, processes for preparing them, and pharmaceutical formulations containing them.

### BACKGROUND TO THE INVENTION

Betamimetics (β-adrenergic substances) are known from the prior art. In this respect, reference may be made, for example, to the disclosure of U.S. Pat. No. 4,341,778 or EP 43940 which proposes betamimetics for the treatment of a wide range of ailments.

For drug treatment of diseases, it is often desirable to prepare medicaments with a longer duration of activity. As a rule, this ensures that the concentration of the active substance in the body needed to achieve the therapeutic effect is maintained for a longer period without the need to re-administer the drug at frequent intervals. Moreover, giving an active substance at longer time intervals contributes to the well-being of the patient to a high degree.

It is particularly desirable to prepare a pharmaceutical composition which can be used therapeutically by administration once a day (single dose). The use of a drug once a day has the advantage that the patient can become accustomed relatively quickly to regularly taking the drug at certain times of the day.

The aim of the present invention is therefore to prepare betamimetics which on the one hand provide a therapeutic benefit in the treatment of respiratory complaints and are also characterised by a longer duration of activity and can thus be used to prepare pharmaceutical compositions with a longer duration of activity. A particular aim of the invention is to prepare betamimetics which, by virtue of their long-lasting effect, can be used to prepare a drug for the treatment of asthma for administration once a day. In addition to these aims, a further objective of the invention is to provide such betamimetics which are not only exceptionally potent but are also characterised by a high degree of selectivity with respect to the β₂-adreno-receptor.

### DETAILED DESCRIPTION OF THE INVENTION

Surprisingly it has been found that the abovementioned problems are solved by compounds of general formula 1.

Accordingly, the present invention relates to the use of one or more, preferably one, compound of general formula 1

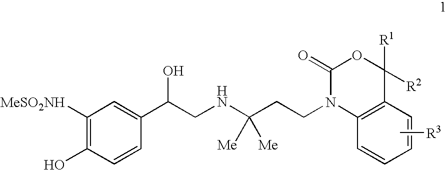

wherein
R¹ and R² which may be identical or different denote hydrogen, halogen, C₁-C₄-alkyl or together denote C₁-C₆-alkylene and
R³ denotes hydrogen, halogen, OH, C₁-C₄-alkyl or —O—C₁-C₄-alkyl;
   for preparing a pharmaceutical composition for the treatment of respiratory complaints which are selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

It is preferable to use for this purpose compounds of general formula 1, wherein
R¹ and R², which may be identical or different, denote hydrogen, fluorine, chlorine, methyl, ethyl, propyl, butyl or together denote —CH₂—CH₂—, —CH₂—CH₂—CH₂—, —CH₂—CH₂—CH₂—CH₂— or —CH₂—CH₂—CH₂—CH₂—CH₂—;
R³ denotes hydrogen, fluorine, chlorine, OH, methyl, ethyl, methoxy, or ethoxy.

It is also preferred to use for the above purpose compounds of general formula 1, wherein
R¹ and R², which may be identical or different, denote hydrogen, methyl, ethyl, propyl or together denote —CH₂—CH₂—, —CH₂—CH₂—CH₂—, —CH₂—CH₂—CH₂—CH₂— or —CH₂—CH₂—CH₂—CH₂—CH₂—;
R³ denotes hydrogen, fluorine, OH, methyl or methoxy.

Preferably the compounds of general formula 1 are used to prepare a pharmaceutical composition for the treatment of obstructive pulmonary diseases selected from the group consisting of bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma or COPD.

Preferably also, the compounds of general formula 1 are used to prepare a pharmaceutical composition for the treatment of pulmonary emphysema which has its origins in COPD or α1-proteinase inhibitor deficiency.

Preferably also, the compounds of general formula 1 are used to prepare a pharmaceutical composition for the treatment of restrictive pulmonary diseases selected from the group consisting of allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

Preferably also, the compounds of general formula 1 are used to prepare a pharmaceutical composition for the treatment of interstitial pulmonary diseases selected from the group consisting of pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

Preferably also, the compounds of general formula 1 are used to prepare a pharmaceutical composition for the treatment of cystic fibrosis or mucoviscidosis.

Preferably also, the compounds of general formula 1 are used to prepare a pharmaceutical composition for the treatment of bronchitis, such as for example bronchitis caused by bacterial or viral infection, allergic bronchitis and toxic bronchitis.

Preferably also, the compounds of general formula 1 are used to prepare a pharmaceutical composition for the treatment of bronchiectasis.

Preferably also, the compounds of general formula 1 are used to prepare a pharmaceutical composition for the treatment of ARDS (adult respiratory distress syndrome).

Preferably also, the compounds of general formula 1 are used to prepare a pharmaceutical composition for the treatment of pulmonary oedema, for example toxic pulmonary oedema after aspiration or inhalation of toxic substances and foreign substances.

Particularly preferably, the present invention relates to the use of the compounds of formula 1 for preparing a pharmaceutical composition for the treatment of asthma. Also of particular importance is the above-mentioned use of compounds of formula 1 for preparing a pharmaceutical composition for once-a-day treatment of inflammatory and obstructive respiratory complaints, particularly for the once-a-day treatment of asthma or COPD.

The present invention also relates to a process for the treatment of the above-mentioned diseases, characterised in that one or more of the above-mentioned compounds of general formula 1 are administered in therapeutically effective amounts. The present invention further relates to processes for the treatment of asthma, characterised in that one or more of the above-mentioned compounds of general formula 1 are administered once a day in therapeutically effective amounts. The present invention further relates to processes for the treatment of COPD, characterised in that one or more of the above-mentioned compounds of general formula 1 are administered once a day in therapeutically effective amounts.

Particularly preferred is the above-mentioned use of compounds of formula 1 which are selected from the group consisting of
N-(5-{2-[1,1-dimethyl-3-(4-methyl-2-oxo-4H-benzo[d][1,3]oxazin-1-yl)- propylamino]-1-hydroxy-ethyl}-2-hydroxy-phenyl)-methanesulphonamide;
N-(5-{2-[1,1-dimethyl-3-(2-oxo-4H-benzo[d][1,3]oxazin-1-yl)-propylamino]-1-hydroxy-ethyl}-2-hydroxy-phenyl)-methanesulphonamide;
N-(5-{2-[3-(4-ethyl-2-oxo-4H-benzo [d][1,3]oxazin-1-yl)-1,1-dimethyl-propylamino]-1-hydroxy-ethyl}-2-hydroxy-phenyl)-methanesulphonamide;
N-(5-{2-[3-(4,4-dimethyl-2-oxo-4H-benzo[d][1,3]oxazin-1-yl)-1,1-dimethyl-propylamino]-1-hydroxy-ethyl}-2-hydroxy-phenyl)-methanesulphonamide;
N-(2-hydroxy-5-{1-hydroxy-2-[3-(6-hydroxy-4,4-dimethyl-2-oxo-4H-benzo[d][1,3]oxazin-1-yl)-1,1-dimethyl-propylamino]-ethyl}-phenyl)-methanesulphonamide;
N-(2-hydroxy-5-{1-hydroxy-2-[3-(6-methoxy-4,4-dimethyl-2-oxo-4H-benzo[d][1,3]oxazin-1-yl)-1,1-dimethyl-propylamino]-ethyl}-phenyl)-methanesulphonamide.

The compounds of formula 1 like the compounds explicitly named above are in some cases known from the prior art. Reference is made particularly in this respect to the disclosure of documents EP 43940 and U.S. Pat. No. 4,341,778.

The present invention further relates to new compounds of formula 1 as such. These are particularly those compounds of formula 1 wherein
R¹ and R² which may be identical or different, preferably identical, denote ethyl or propyl, or together denote —CH₂—CH₂—, —CH₂—CH₂—CH₂—, —CH₂—CH₂—CH₂—CH₂— or —CH₂—CH₂—CH₂—CH₂—CH₂— and wherein
R³ may denote hydrogen, fluorine, chlorine, OH, methyl, ethyl, methoxy or ethoxy.

Particularly preferred are the compounds of formula 1 wherein
R¹ and R² which may be identical or different, preferably identical, denote ethyl or propyl, or together denote —CH₂—CH₂—, —CH₂—CH₂—CH₂—CH₂— or —CH₂—CH₂—CH₂—CH₂—CH₂— and wherein
R³ may denote hydrogen, fluorine, OH, methyl or methoxy, preferably hydrogen.

Particularly preferred are the compounds of formula 1 wherein
R¹ and R² are identical and represent ethyl or propyl and wherein
R³ may denote hydrogen, fluorine, OH, methyl or methoxy, preferably hydrogen.

Particularly preferred are the compounds of formula 1, wherein
R¹ and R² together denote —CH₂—CH₂—, —CH₂—CH₂—CH₂—CH₂— or —CH₂—CH₂—CH₂—CH₂—CH₂— and wherein
R³ may denote hydrogen, fluorine, OH, methyl or methoxy, preferably hydrogen.

Particularly preferred are the compounds of formula 1, wherein R³ denotes hydrogen and R¹ and R² may have the meanings given above.

Particularly preferred are the compounds of formula 1, wherein R³ denotes fluorine and R¹ and R² may have the meanings given above.

Particularly preferred are the compounds of formula 1, wherein
R¹ and R² both represent ethyl or propyl and wherein R³ may have the meanings given above.

Particularly preferred are those compounds of formula 1 which are selected from the group consisting of
N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide;
N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide;
N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide;
N-(5-{2-[3-(4,4-diethyl-2-oxo-4H-benzo [d][1,3]oxazin-1-yl)-1,1-dimethyl-propylamino]-1-hydroxy-ethyl}-2-hydroxy-phenyl)-methanesulphonamide;
N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide;
N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide;
N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide;
N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide.

In another aspect the present invention relates to the above-mentioned new compounds of formula 1 as pharmaceutical compositions. The present invention also relates to the use of the above-mentioned new compounds of formula 1 for preparing a pharmaceutical composition for the treatment of respiratory complaints which are selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

In another aspect the present invention relates to the above-mentioned use of the compounds of formula 1 in the form of the individual optical isomers, mixtures of the individual enantiomers, diastereomers or racemates. Particularly preferred is the above-mentioned use of the compounds of formula 1 in the form of the enantiomerically or diastereomerically pure compounds, while the use of the R-enantiomers of the compounds of formula R-1

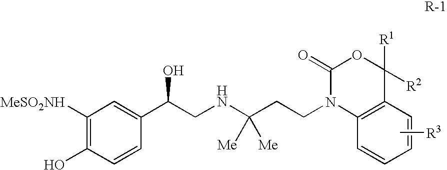

wherein the groups R¹, R² and R³ may have the meanings given above, is of exceptional importance according to the invention.

In another aspect the present invention relates to the above-mentioned new compounds of formula 1 in the form of the individual optical isomers, mixtures of the individual enantiomers, diastereomers or racemates. Particularly preferred are the above-mentioned new compounds of formula 1 in the form of the enantiomerically or diastereomerically pure compounds, while the R-enantiomer of formula R-1 is of exceptional importance according to the invention.

In another aspect the present invention relates to the above-mentioned use of the compounds of formula 1 in the form of the free bases or in the form of the acid addition salts with pharmacologically acceptable acids, as well as optionally in the form of the solvates and/or hydrates.

In another aspect the present invention relates to the above-mentioned new compounds of formula 1 in the form of the free bases or in the form of the acid addition salts with pharmacologically acceptable acids, as well as optionally in the form of the solvates and/or hydrates.

By acid addition salts with pharmacologically acceptable acids are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Of the above-mentioned acid addition salts the salts of hydrochloric acid, methanesulphonic acid, benzoic acid and acetic acid are particularly preferred according to the invention.

By alkyl groups are meant, unless otherwise stated, branched and unbranched alkyl groups with 1 to 4 carbon atoms. The following are mentioned by way of example: methyl, ethyl, propyl or butyl. The abbreviations Me, Et, Prop or Bu may also optionally be used to denote the groups methyl, ethyl, propyl or butyl. Unless stated otherwise, the definitions propyl and butyl include all the possible isomeric forms of the groups in question. Thus, for example, propyl includes n-propyl and iso-propyl, butyl includes iso-butyl, sec. butyl and tert.-butyl etc.

Suitable alkylene groups, unless otherwise stated, are branched and unbranched doubly-bound alkyl bridges with 1 to 6 carbon atoms. The following are mentioned by way of example: methylene, ethylene, n-propylene or n-butylene.

Examples of alkyloxy groups (or also —O-alkyl groups) are, unless otherwise stated, branched and unbranched alkyl groups with 1 to 4 carbon atoms which are linked via an oxygen atom. The following are mentioned by way of example: methyloxy, ethyloxy, propyloxy or butyloxy. The abbreviations MeO—, EtO—, PropO— or BuO— may also optionally be used to denote the groups methyloxy, ethyloxy, propyloxy or butyloxy. Unless stated otherwise, the definitions propyloxy and butyloxy include all the possible isomeric forms of the groups in question. Thus, for example, propyloxy includes n-propyloxy and iso-propyloxy, butyloxy includes iso-butyloxy, sec. butyloxy and tert.-butyloxy etc. Optionally, within the scope of the present invention, the term alkoxy is used instead of the term alkyloxy. The terms methoxy, ethoxy, propoxy or butoxy may optionally also be used to denote the groups methyloxy, ethyloxy, propyloxy or butyloxy.

Halogen within the scope of the present invention denotes fluorine, chlorine, bromine or iodine. Unless stated to the contrary, fluorine, chlorine and bromine are regarded as preferred halogens.

The preparation of the compounds according to the invention may be carried out according to or analogously to procedures already known in the prior art. Suitable methods of production are known for example from EP43940 or U.S. Pat. No. 4,341,778, the entire contents of which are herein incorporated by reference.

The Examples described below serve to further illustrate some compounds known from the prior art which may surprisingly be used according to the present invention to treat the above-mentioned respiratory complaints.

### EXAMPLES

### Example 1

### N-(5-{2-[1,1-dimethyl-3-(4-methyl-2-oxo-4H-benzo[d]1,3]oxazin-1-yl)-propylaminol-1-hydroxy-ethyl}-2-hydroxy-phenyl)-methanesulphonamide

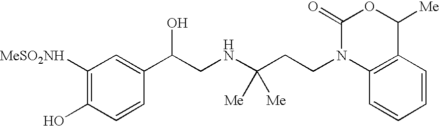

The compound is known from EP 43940. The individual diastereomers of this embodiment may be obtained by common methods known in the art.

### Example 2

### N-(5-{2-[1,1-dimethyl-3-(2-oxo-4H-benzo[d][1,3]oxazin-1-yl)-propylamino]-1-hydroxy-ethyl}-2-hydroxy-phenyl)-methanesulphonamide

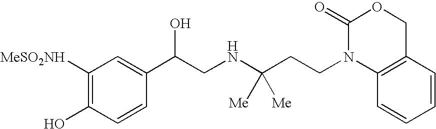

The compound is known from EP 43940. The (R)- and (S)-enantiomers of this embodiment may be obtained by common methods known in the art.

### Example 3

### N-(5-{2-[3-(4-ethyl-2-oxo-4H-benzo[d][1,3]oxazin-1-yl)-1,1-dimethyl-propylamino]-1-hydroxy-ethyl}-2-hydroxy-phenyl)-methanesulphonamide

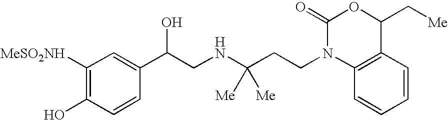

The compound is known from EP 43940. The individual diastereomers of this embodiment may be obtained by common methods known in the art.

### Example 4

### N-(5-{2-[3-(4,4-dimethyl-2-oxo-4H-benzo[d][1,3]oxazin-1-yl)-1,1-dimethyl-propylamino]-1-hydroxy-ethyl}-2-hydroxy-phenyl)-methanesulphonamide

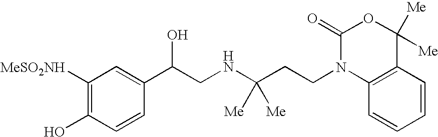

The compound is known from EP 43940. The (R)- and (S)-enantiomers of this embodiment may be obtained by common methods known in the art.

### Example 5

### N-(2-hydroxy-5-{1-1-hydroxy-2-[3-(6-hydroxy-4,4-dimethyl-2-oxo-4H-benzo[d][1,3]oxazin-1-yl)-1,1-dimethyl-propylamino]-ethyl}-phenyl)-methanesulphonamide

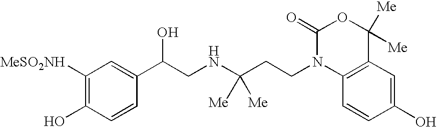

The compound is known from EP 43940. The (R)- and (S)-enantiomers of this embodiment may be obtained by common methods known in the art.

### Example 6

### N-(2-hydroxy-5-{1-hydroxy-2-[3-(6-methoxy-4,4-dimethyl-2-oxo-4H-benzo[d][1,3]oxazin-1-yl)-1,1-dimethyl-propylamino]-ethyl}-phenyl)-methanesulphonamide

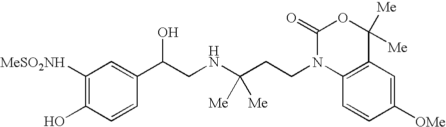

The compound is known from EP 43940. The (R)- and (S)-enantiomers of this embodiment may be obtained by common methods known in the art.

The examples of synthesis described below serve as further illustration of new compounds according to the invention. They are only meant, however, as examples of procedures to illustrate the invention further without restricting it to the subject matter described by way of example hereinafter.

HPLC method (method A): Symmetry C18 (Waters): 3.5 μm; 4.6×150 mm; column temperature: 20° C.; gradient: acetonitrile/phosphate buffer (pH 7) 20:80→80:20 in 30 minutes; flow: 1.0 mL/min; detection at 220 and 254 nm.

### Synthesis of intermediate products 1-8

### Intermediate product 1: 1-(3-amino-3-methyl-butyl)-4,4-dipropyl-1,4-dihydro-benzo[d][1,3]oxazin-2-one

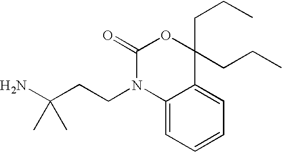

a) 4-(2-amino-phenyl)-heptan-4-ol

90 mL (180.0 mmol) propylmagnesium chloride (2 M in ether) are added dropwise at 0° C. within 30 minutes to a solution of 7.00 mL (54.0 mmol) methyl anthranilate in abs. THF (70 mL). The mixture is stirred for one hour at ambient temperature and then combined with 100 mL 3 molar aqueous ammonium chloride solution and ethyl acetate. The phases are separated and the aqueous phase is exhaustively extracted with ethyl acetate. The combined organic phases are washed with potassium hydrogen carbonate solution and saturated sodium chloride solution and dried with sodium sulphate. The crude product is used in the next reaction step without any further purification. Yield: 6.70 g (60%).

b) tert-butyl {3-[2-(1-hydroxy-1-propyl-butyl)-phenylamino]-1,1-dimethyl-propyl}-carbamate

1.40 g (22.27 mmol) sodium cyanoborohydride are added to a solution of 3.10 g (14.05 mmol) 4-(2-amino-phenyl)-heptan-4-ol and 3.60 g (17.88 mmol) tert-butyl (1,1-dimethyl-3-oxo-propyl)-carbamate in methanol (40 mL) and acetic acid (6 mL). The mixture is stirred for 16 hours at ambient temperature, diluted with ethyl acetate, washed with 0.5 molar potassium hydrogen sulphate solution and saturated sodium chloride solution, dried with sodium sulphate and evaporated down in vacuo. The crude product is used in the next reaction step without any further purification. Yield: 6.00 g (quantitative yield).

c) tert-butyl [1,1-dimethyl-3-(2-oxo-4,4-dipropyl-4H-benzo[d][1,3]oxazin-1-yl)-propyl]-carbamate

8.85 mL (16.81 mmol) phosgene solution (20 wt. % in toluene) are slowly added dropwise at 0° C. to a solution of 6.00 g (15.28 mmol) tert-butyl {3-[2-(1-hydroxy-1-propyl-butyl)-phenylamino]-1,1-dimethyl-propyl}-carbamate and 5.32 mL (38.21 mmol) triethylamine in abs. THF (80 mL). The mixture is stirred for 2 hours at ambient temperature, diluted with ethyl acetate, combined with ice and made basic with saturated aqueous ammonia solution. The aqueous phase is exhaustively extracted with ethyl acetate and the combined organic phases are washed with saturated sodium chloride solution, dried with sodium sulphate and evaporated down in vacuo. After column chromatography (silica gel, cyclohexane/ethyl acetate=6:1) the product is obtained as a yellow oil. Yield: 4.57 g (71%).

d) 1-(3-amino-3-methyl-butyl)-4,4-dipropyl-1,4-dihydro-benzo[d][1,3]oxazin-2-one

A solution of 4.20 g (10.03 mmol) tert-butyl [1,1-dimethyl-3-(2-oxo-4,4-dipropyl-4H-benzo[d][1,3]oxazin-1-yl)-propyl]-carbamate in 35 mL formic acid is stirred for 24 hours at ambient temperature and then poured onto ice. The aqueous phase is made basic with saturated aqueous ammonia solution and exhaustively extracted with ethyl acetate. The combined organic extracts are washed with sodium chloride solution, dried with sodium sulphate and evaporated down in vacuo. The residue is taken up in ethyl acetate (50 mL) and combined with 4 mL hydrochloric acid in ethyl acetate (saturated). The solution is concentrated by evaporation and combined twice with a little ethanol and evaporated down in vacuo. Trituration of the residue with diisopropylether yields the product as a hygroscopic hydrochloride salt. Yield: 2.60 g (73%).

Intermediate product 2: 1-(3-amino-3-methyl-butyl)-4,4-diethyl-7-fluoro-1,4-dihydro-benzo[d][1,3]oxazin-2-one

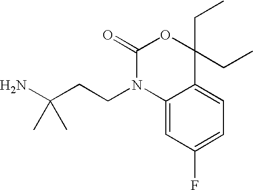

a) 3-(2-amino-4-fluoro-phenyl)-pentan-3-ol

The product is obtained analogously to intermediate product 1a by reaction of methyl 2-amino-4-fluoro-benzoate and ethylmagnesium bromide in dichloromethane at −78° C. with heating to ambient temperature. Yield: 4.1 g (99%).

b) tert-butyl {3-[2-(1-ethyl-1-hydroxy-propyl)-5-fluoro-phenylamino]-1,1-dimethyl-propyl}-carbamate

The product is obtained analogously to intermediate product 1b starting from 3-(2-amino-4-fluoro-phenyl)-pentan-3-ol and tert-butyl (1,1-dimethyl-3-oxo-propyl)-carbamate. The crude product is purified by column chromatography (silica gel, dichloromethane/methanol=100:0→98:2). Yield: 7.70 g (99%).

c) tert-butyl[3-(4,4-diethyl-7-fluoro-2-oxo-4H-benzo[d][1,3]oxazin-1-yl)-1,1-dimethyl-propyl]-carbamate

The product is obtained analogously to intermediate product 1c starting from tert-butyl {3-[2-(1-ethyl-1-hydroxy-propyl)-5-fluoro-phenylamino]-1,1-dimethyl-propyl}-carbamate. Yield: 4.20 g (51%).
d) 1-(3-amino-3-methyl-butyl)-4,4-diethyl-7-fluoro-1,4-dihydro-benzo[d][1,3]oxazin-2-one

The product is prepared as the free base analogously to intermediate product 1d starting from tert-butyl [3-(4,4-diethyl-7-fluoro-2-oxo-4H-benzo[d][1,3]oxazin-1-yl)-1,1-dimethyl-propyl]-carbamate. Yield: 2.90 g (96%); ESI-MS: [M+H]⁺=309.

Intermediate product 3: 1-(3-amino-3-methyl-butyl)-spiro(cyclopropyl-1,4′-2H-3′,1′-benzoxazin)-2′-one

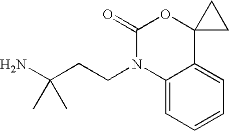

a) 1-(2-dibenzylamino-phenyl)-cyclopropanol

2.45 mL (8.4 mmol) titanium tetraisopropoxide are slowly added dropwise at ambient temperature to a solution of 18.5 g (55.8 mmol) methyl 2-dibenzylamino-benzoate in 150 mL THF. After one hour's stirring 40.9 mL (122.7 mmol) ethylmagnesium bromide (3 M in diethyl ether) are added. The mixture is stirred for one hour, a further 4 mL of 3 molar ethylmagnesium bromide solution are added and the mixture is stirred for 2 hours. The reaction mixture is combined with saturated ammonium chloride solution and extracted with ethyl acetate. The aqueous phase is combined with 1 molar hydrochloric acid until a clear solution is obtained and extracted with ethyl acetate. The combined organic phases are washed with sodium hydrogen carbonate solution and sodium chloride solution, dried with sodium sulphate and evaporated down. The residue is purified by chromatography (hexane/ethyl acetate=20:1). Yellow oil. Yield: 10.0 g (54%).

b) 1-(2-amino-phenyl)-cyclopropanol

9.90 g (30.1 mmol) 1-(2-dibenzylamino-phenyl)-cyclopropanol are dissolved in 70 mL methanol and hydrogenated in the presence of 1 g palladium on charcoal (10%) at 3 bar hydrogen pressure. The catalyst is suction filtered, the filtrate is evaporated down and the residue is purified by chromatography (silica gel; cyclohexane/ethyl acetate=5:1). White solid. Yield: 1.80 g (40%).

c) tert-butyl {3-[2-(1-hydroxy-cyclopropyl)-phenylamino]-1,1-dimethyl-propyl}-carbamate

Prepared analogously to the method described for intermediate product 1b from 1.77 g (11.86 mmol) 1-(2-amino-phenyl)-cyclopropanol and 3.15 g (15.66 mmol) tert-butyl (1,1-dimethyl-3-oxo-propyl)-carbamate. The crude product obtained is purified by column chromatography (silica gel, cyclohexane/ethyl acetate 4:1) purified. Yellow oil. Yield: 2.60 g.

d) tert-butyl {1,1-dimethyl-3-[spiro(cyclopropyl-14′-2H-3′,1′-benzoxazin)-2′-oxo-1-yl]-propyl}-carbamate

The product is obtained analogously to intermediate product 1c starting from 2.60 g (7.74 mmol) tert-butyl {3-[2-(1-hydroxy-cyclopropyl)-phenylamino]-1,1-dimethyl-propyl}-carbamate. However, no purification by column chromatography is carried out. Yellow oil. Yield: 2.60 g.

e) 1-(3-amino-3-methyl-butyl)-spiro(cyclopropyl-1,4′-2H-3′,1′-benzoxazin)-2′-one

Obtained analogously to the method described for intermediate stage 1d from the reaction of 3.10 g (8.60 mmol) tert-butyl {1,1-dimethyl-3-[spiro(cycloproyl-1,4′-2H-3′,1′-benzoxazin)-2′-oxo-1-yl]-propyl}-carbamate and 30 mL formic acid. Yellow oil. Yield: 2.10 g (94%).

Intermediate product 4: 1-(3-amino-3-methyl-butyl)-4,4-diethyl-1,4-dihydro-benzo[d][1,3]oxazin-2-one

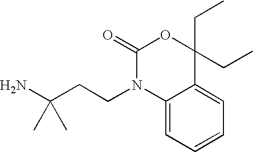

a) 3-(2-amino-phenyl)-pentan-3-ol

100 mL of a 3 molar ethylmagnesium bromide solution in diethyl ether are added dropwise at −40° C. to a solution of 7.77 mL (60 mmol) 2-amino-methylbenzoic acid in 130 mL THF. The mixture is stirred overnight with heating to ambient temperature, combined with saturated ammonium chloride solution, acidified with 1 molar hydrochloric acid and extracted with ethyl acetate. The combined organic phases are extracted with water, dried with sodium sulphate and evaporated down. Dark red oil which crystallises out and is further reacted directly. Yield: 10.9 g; mass spectroscopy: [M+H]⁺=180.

b) tert-butyl {3-[2-(1-ethyl-1-hydroxy-propyl)-phenylamino]-1,1-dimethyl-propyl}-carbamate

3.16 g (47.7 mmol) sodium cyanoborohydride are added at ambient temperature to 5.70 g (31.8 mmol) 3-(2-amino-phenyl)-pentan-3-ol and 2.63 mL (47.7 mmol) acetic acid in 18 mL methanol. Then a solution of 7.04 g (35 mmol) tert-butyl (1,1-dimethyl-3-oxo-propyl)-carbamate in 18 mL methanol is slowly added dropwise. After the addition has ended the mixture is stirred for four hours, combined with 1 molar hydrochloric acid (development of gas) and then made basic with aqueous ammonia solution. It is extracted with ethyl acetate and the combined organic phases are washed with sodium chloride solution, dried with sodium sulphate and freed from the solvent. The residue is purified by column chromatography (silica gel, dichloromethane/methanol gradient with 0.1% ammonia). Yellow oil. Yield: 4.25 g (37%); mass spectroscopy: [M+H]⁺=365.

c) tert-butyl [3-(4,4-diethyl-2-oxo-4H-benzo[d][1,3]oxazin-1-yl)-1,1-dimethyl-propyl]-carbamate

2.91 g (9.6 mmol) triphosgene are added to a solution of 3.50 g (9.6 mmol) tert-butyl {3-[2-(1-ethyl-1-hydroxy-propyl)-phenylamino]-1,1-dimethyl-propyl}-carbamate and 3.37 mL (24 mmol) triethylamine in 35 mL THF at 0 to 5° C. The mixture is stirred overnight at ambient temperature and the precipitate formed is suction filtered. The filtrate is evaporated down and the oil remaining is further reacted directly. Yield: 3.33 g; mass spectroscopy: [M+H]⁺=391.

d) 1-(3-amino-3-methyl-butyl)-4,4-diethyl-1,4-dihydro-benzo[d][1,3]oxazin-2-one

25 mL trifluoroacetic acid are added dropwise to a solution of 3.20 g tert-butyl [3-(4,4-diethyl-2-oxo-4H-benzo [d][1,3]oxazin-1-yl)-1,1-dimethyl-propyl]-carbamate (approx. 75%) in 25 mL dichloromethane while being cooled with the ice bath. The mixture is stirred for 2 hours at ambient temperature, the solvents are distilled off and acid residues are removed by repeated co-distillation with toluene. To liberate the free base the residue is combined with 1 molar sodium hydroxide solution and extracted with ethyl acetate. The organic phases are dried with sodium sulphate and evaporated down. The free base is dissolved in 8 mL methanol and combined with ethereal hydrochloric acid. It is stirred overnight and the precipitate formed is suction filtered and washed with diethyl ether. Yield: 2.15 g (hydrochloride); mass spectroscopy: [M+H]⁺=291.

Intermediate product 5: 1-(3-amino-3-methyl-butyl)-spiro(cyclohexane-1,4′-2H-3′,1′-benzoxazin)-2′-one

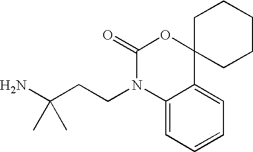

a) 1-(2-nitro-phenyl)-cyclohexanol

40.16 mL (80.32 mmol) phenylmagnesium chloride (2 M in THF) are added dropwise to a solution of 20.0 g (80.32 mmol) 2-nitro-iodobenzene in 150 mL THF at −50° C. under nitrogen. After 15 minutes stirring 9.98 mL (96.30 mmol) cyclohexanone are added quickly. The reaction mixture is heated to ambient temperature, stirred for two hours and combined with ammonium chloride solution. The aqueous phase is separated off and exhaustively extracted with ethyl acetate. The combined organic phases are washed with sodium chloride solution, dried with sodium sulphate and evaporated down. Column chromatography (silica gel, hexane/ethyl acetate=20:1) yields the product as a brownish oil. Yield: 5.20 g (29%); Rₜ=0.26 (silica gel, hexane/ethyl acetate=10:1); ESI-MS: [M+H-H₂O]⁺=204.

b) 1-(2-amino-phenyl)-cyclohexanol

5.20 g (16.45 mmol) 1-(2-nitro-phenyl)-cyclohexanol in 70 mL ethanol are hydrogenated in the presence of Raney nickel at ambient temperature and 3 bar hydrogen pressure for 4 hours. The catalyst is filtered off through Celite and the filtrate is evaporated down in vacuo. The residue is precipitated from hexane. Yield: 1.53 g (49%); Rₜ=0.38 (silica gel, hexane/ethyl acetate=4:1); ESI-MS: [M+H−H₂O]⁺=174.

c) tert-butyl {3-[2-(1-hydroxy-cyclohexyl)-phenylamino]-1,1-dimethyl-propyl}-carbamate

The compound is obtained analogously to intermediate product 1b from 1-(2-aminophenyl)-cyclohexanol and tert-butyl (1,1-dimethyl-3-oxo-propyl)-carbamate. Column chromatography (silica gel, hexane/ethyl acetate=7:1) yields the product in the form of a colourless oil. Yield: 2.65 g (66%); Rₜ=0.50 (silica gel, hexane/ethyl acetate=4:1).

d) tert-butyl {1,1-dimethyl-3-[spiro(cyclohexane-1,4′-2H-3′,1′-benzoxazin)-2′-oxo-1-yl]-propyl}-carbamate

Prepared analogously to intermediate product 1c from tert-butyl {3-[2-(1-hydroxy-cyclohexyl)-phenylamino]-1,1-dimethyl-propyl}-carbamate. Yield: 2.60 g (92%); Rₜ=0.38 (silica gel, hexane/ethyl acetate 4:1).

e) 1-(3-amino-3-methyl-butyl)-spiro(cyclohexane-1,4′-2H-3′,1′-benzoxazin)-2′-one

Prepared analogously to intermediate product 1d from tert-butyl [1,1-dimethyl-3-(spiro(cyclohexane-1,4′-2H-3′,1′-benzoxazin)-2′-oxo-1-yl)-propyl]-carbamate. Yield: 1.80 g (92%); Rₜ=0.10 (silica gel, dichloromethane/methanol/ammonia=95:5:0.5); ESI-MS: [M+H]⁺=303.

Intermediate product 6: 1-(3-amino-3-methyl-butyl)-4,4-diethyl-8-methoxy-1,4-dihydro-benzo[d][1,3]oxazin-2-one

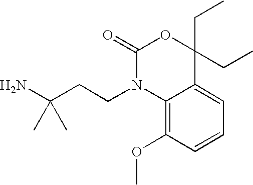

a) 3-(2-amino-3-methoxy-phenyl)-pentan-3-ol

The product is obtained analogously to intermediate product 1a by reacting methyl 2-amino-3-methoxy-benzoate and ethylmagnesium bromide in dichloromethane at −78° C.→RT. Yield: 5.20 g (92%); HPLC-MS: Rₜ=12.85 min. (method A); ESI-MS: [M+H]⁺=210.

b) tert-butyl {3-[2-(1-ethyl-1-hydroxy-propyl)-6-methoxy-phenylamino]-1,1-dimethyl-propyl}-carbamate

The product is obtained analogously to intermediate product 1b starting from 3-(2-amino-3-methoxy-phenyl)-pentan-3-ol and tert-butyl (1,1-dimethyl-3-oxo-propyl)-carbamate. The crude product is purified by column chromatography (silica gel, cyclohexane/ethyl acetate=4:1). Yield: 4.60 g (47%).

c) tert-butyl [3-(4,4-diethyl-8-methoxy-2-oxo-4H-benzo[d][1,3]oxazin-1-yl)-1,1-dimethyl-propyl]-carbamate

The product is obtained analogously to intermediate product 1c starting from tert-butyl {3-[2-(1-ethyl-1-hydroxy-propyl)-6-methoxy-phenylamino]-1,1-dimethyl-propyl}-carbamate. Yield: 4.60 g (94%).

d) 1-(3-amino-3-methyl-butyl)-4,4-diethyl-8-methoxy-1,4-dihydro-benzo[d][1,3]oxazin-2-one

The product is obtained analogously to intermediate product 1d starting from tert-butyl [3-(4,4-diethyl-8-methoxy-2-oxo-4H-benzo[d][1,3]oxazin-1-yl)-1,1-dimethyl-propyl]-carbamate as the free base. Yield: 3.00 g (93%); ESI-MS: [M+H]⁺=321.

Intermediate product 7: 1-(3-amino-3-methyl-butyl)-4,4-diethyl-6-fluoro-1,4-dihydro-benzo[d][1,3]oxazin-2-one

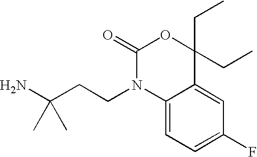

a) 3-(2-amino-5-fluoro-phenyl)-pentan-3-ol

Prepared analogously to intermediate product la from methyl 2-amino-5-fluoro-benzoate and ethylmagnesium bromide. The product obtained is purified by chromatography (silica gel, cyclohexane/ethyl acetate=8:1). Yield: 6.00 g (74%).

b) tert-butyl {3-[2-(1-ethyl-1-hydroxy-propyl)-4-fluoro-phenylamino]-1,1-dimethyl-propyl}-carbamate

The product is obtained analogously to intermediate product 1b starting from 3-(2-amino-5-fluoro-phenyl)-pentan-3-ol and tert-butyl (1,1-dimethyl-3-oxo-propyl)-carbamate. The crude product is purified by column chromatography (silica gel, hexane/ethyl acetate=6:1→2:1). Yield: 4.50 g (41%).

c) tert-butyl [3-(4,4-diethyl-6-fluoro-2-oxo-4H-benzo[d][1,3]oxazin-1-yl)-1,1-dimethyl-propyl]-carbamate

Prepared analogously to intermediate product 1c from tert-butyl {3-[2-(1-ethyl-1-hydroxy-propyl)-4-fluoro-phenylamino]-1,1-dimethyl-propyl}-carbamate. However, in this case no purification by column chromatography is carried out. Colourless oil. Yield: 4.8 g.

d) 1-(3-amino-3-methyl-butyl)-4,4-diethyl-6-fluoro-1,4-dihydro-benzo[d][1,3]oxazin-2-one

The target compound is prepared as the free base analogously to intermediate product 1d from tert-butyl [3-(4,4-diethyl-6-fluoro-2-oxo-4H-benzo[d][1,3]oxazin-1-yl)-1,1-dimethyl-propyl]-carbamate. Yield: 3.00 g (99%).

Intermediate product 7: 1-(3-amino-3-methyl-butyl)-4,4-diethyl-6-methoxy-1,4-dihydro-benzo[d][1,3]oxazin-2-one

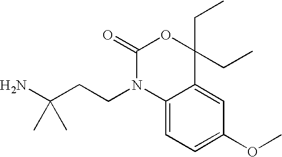

a) 3-(2-amino-5-methoxy-phenyl)-pentan-3-ol

The product is obtained by reacting 4.00 g (22 mmol) methyl 2-amino-5-methoxy-benzoate with 5 equivalents of ethylmagnesium bromide in dichloromethane at −78° C.→RT. Brown oil. Yield: 4.47 g (97%).

b) tert-butyl {3-[2-(1-ethyl-1-hydroxy-propyl)-4-methoxy-phenylamino]-1,1-dimethyl-propyl}-carbamate

Prepared analogously to intermediate product 1b from 4.45 g (21 mmol) 3-(2-amino-5-methoxy-phenyl)-pentan-3-ol and 5.66 g (28 mmol) tert-butyl (1,1-dimethyl-3-oxo-propyl)-carbamate. Brown oil. Yield: 6.00 g (72%).

c) tert-butyl [3-(4,4-diethyl-6-methoxy-2-oxo-4H-benzo[d][1,3]oxazin-1-yl)-1,1-dimethyl-propyl]-carbamate

The product is prepared analogously to intermediate product 1c from 6.00 g (15.2 mmol) tert-butyl {3-[2-(1-ethyl-1-hydroxy-propyl)-4-methoxy-phenylamino]-1,1-dimethyl-propyl}-carbamate. Yellow oil. Yield: 3.10 g (48%).

d) 1-(3-amino-3-methyl-butyl)-4,4-diethyl-6-methoxy-1,4-dihydro-benzo[d][1,3]oxazin-2-one

Prepared analogously to intermediate product 1d from 3.10 g (8.5 mmol) tert-butyl [3-(4,4-diethyl-6-methoxy-2-oxo-4H-benzo [d][1,3] oxazin-1-yl)-1,1-dimethyl-propyl]-carbamate. The product is isolated as the free base and not converted into a hydrochloride salt. Yellow oil. Yield: 2.20 g (98%).

### Example 7

### N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide

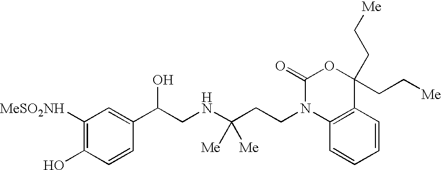

a) N-(2-benzyloxy-5-{2-[1,1-dimethyl-3-(2-oxo-4,4-dipropyl-4H-benzo[d][1,3]oxazin-1-yl)-propylamino]-1-hydroxy-ethyl}-phenyl)-methanesulphonamide

86 μl (0.619 mmol) triethylamine are added at ambient temperature under a nitrogen atmosphere to a solution of 200 mg (0.564 mmol) 1-(3-amino-3-methyl-butyl)-4,4-dipropyl-1,4-dihydro-benzo[d][1,3]oxazin-2-one hydrochloride in 5 mL THF. The mixture is stirred for 30 minutes, 218 mg (0.575 mmol) N-[2-benzyloxy-5-(2-ethoxy-2-hydroxy-acetyl)-phenyl]-methanesulphonamide are added and the mixture is stirred for a further 2 hours at ambient temperature. It is cooled to 10° C., combined with 51 mg (2.34 mmol) lithium borohydride, heated to ambient temperature and stirred for one hour. It is again cooled to 10° C. and diluted with 15 mL water and 20 mL dichloromethane. The aqueous phase is separated off and extracted with dichloromethane. The combined organic phases are dried with sodium sulphate and evaporated down in vacuo. The residue is dissolved in 8 mL ethyl acetate and acidified to pH 2 by the addition of saturated hydrochloric acid in ethyl acetate. The precipitate that forms is filtered off, washed with ethyl acetate and evaporated down. Yield: 260 mg (67%, hydrochloride), HPLC: Rₜ=19.8 minutes (method A).

b) N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide

260 mg (0.386 mmol) N-(2-benzyloxy-5-{2-[1,1-dimethyl-3-(2-oxo-4,4-dipropyl-4H-benzo [d][1,3] oxazin-1-yl)-propylamino]-1-hydroxy-ethyl}-phenyl)-methanesulphonamide hydrochloride in 8 mL methanol are hydrogenated in the presence of 26 mg palladium on charcoal (10%) at ambient temperature. The catalyst is filtered off through Celite and washed with methanol. The filtrate is evaporated down in vacuo and the residue is stirred in diethyl ether. Yield: 120 mg (53%, hydrochloride); mass spectroscopy: [M+H]⁺=548; HPLC: Rₜ=14.7 minutes (method A).

The (R)- and (S)-enantiomers of this embodiment may be obtained by common methods known in the art. The (R)-enantiomer of this embodiment has particular importance according to the invention.

### Example 8

### N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide

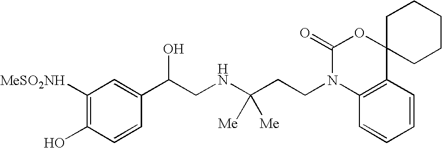

a) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide

Prepared analogously to the process described for Example 7a from 250 mg (0.66 mmol) N-[2-benzyloxy-5-(2-ethoxy-2-hydroxy-acetyl)-phenyl]-methanesulphonamide and 200 mg (0.66 mmol) 1-(3-amino-3-methyl-butyl)-spiro(cyclohexane-1,4′-2H-3′,1′-benzoxazin)-2′-one. However, the difference is that the product obtained as the hydrochloride is also purified by chromatography (silica gel, dichloromethane/methanol=50:1). Yield: 190 mg (46%), HPLC: Rₜ=17.8 minutes (method A).

b) N-[5-(2-{1,1-dimethyl-3-[spiro(cyclohexane-1,4′-2H-3′ 1′-benzoxazin)-2′-oxo-1-yl]-propylamino}-1-hydroxy-ethyl)-2-hydroxy-phenyl]-methanesulphonamide

190 mg (0.31 mmol) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide are hydrogenated analogously to Example 7b. After removal of the catalyst the filtrate is freed from the solvent, combined with 8 mL ethyl acetate and acidified to pH 2 by the addition of hydrochloric acid in ethyl acetate. The solvent is distilled off and the residue is stirred in diethyl ether and filtered. Yield: 40 mg (23%, hydrochloride); mass spectroscopy: [M+H]⁺=532; HPLC: Rₜ=11.8 minutes (method A).

The (R)- and (S)-enantiomers of this embodiment may be obtained by common methods known in the art. The (R)-enantiomer of this embodiment has particular importance according to the invention.

### Example 9

### N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide

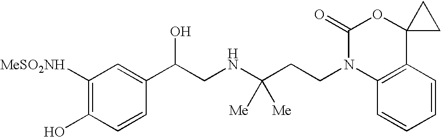

a) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide

292 mg (0.77 mmol) N-[2-benzyloxy-5-(2-ethoxy-2-hydroxy-acetyl)-phenyl]-methanesulphonamide and 200 mg (0.77 mmol) 1-(3-amino-3-methyl-butyl)-spiro(cyclopropyl-1,4′-2H-3′,1′-benzoxazin)-2′-one are reacted and worked up analogously to Example 7a. The crude product is combined with 8 mL ethyl acetate and acidified to pH 2 with hydrochloric acid in ethyl acetate. The solvent is distilled off and the residue is stirred in diethyl ether. White solid. Yield: 400 mg (84%, hydrochloride), HPLC: Rₜ=15.2 minutes (method A).

b) N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide

The product is prepared analogously to Example 1b from 400 mg (0.65 mmol) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide hydrochloride. Yield: 230 mg (67%, hydrochloride); mass spectroscopy: [M+H]⁺=490; HPLC: Rₜ=8.9 minutes (method A).

The (R)- and (S)-enantiomers of this embodiment may be obtained by common methods known in the art. The (R)-enantiomer of this embodiment has particular importance according to the invention.

### Example 10

### N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide

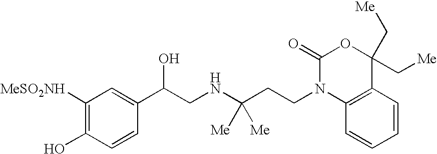

379 mg (1 mmol) N-[2-benzyloxy-5-(2-ethoxy-2-hydroxy-acetyl)-phenyl]-methanesulphonamide and 290 mg (1 mmol) 1-(3-amino-3-methyl-butyl)-4,4-diethyl-1,4-dihydro-benzo[d][1,3]oxazin-2-one are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for a further 30 minutes. The mixture is filtered through aluminium oxide (basic), washed repeatedly with dichloromethane/methanol (15:1) and evaporated down. The crude product thus obtained is purified by chromatography (dichloromethane with 0-10% methanol/ammonia=9:1). The benzylether thus obtained is dissolved in 10 mL methanol and hydrogenated with palladium on charcoal as catalyst at 1 bar hydrogen pressure. Then the catalyst is filtered off and the filtrate is evaporated down. White solid. Yield: 338 mg (65% over 2 steps); mass spectroscopy: [M+H]⁺=520.

The (R)- and (S)-enantiomers of this embodiment may be obtained by common methods known in the art. The (R)-enantiomer of this embodiment has particular importance according to the invention. The rotational value of (R)-N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide hydrochloride (co-crystallised with one molecule of acetone) is −28.8° (c=1%, in methanol at 20° C.).

### Example 11

### N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide

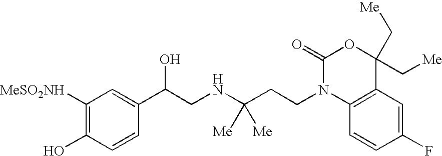

a) N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide

Reaction of 246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-6-fluoro-1,4-dihydro-benzo [D][1,3]oxazin-2-one analogously to Example 7a. However, the preparation of the hydrochloride is omitted. Instead, the free base is purified by chromatography (reverse phase, acetonitrile/water gradient with 0.1% trifluoroacetic acid). Yield: 180 mg (trifluoroacetate), HPLC: Rₜ=17.4 minutes (method A).

b) N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide

175 mg N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide trifluoroacetate in 9 mL methanol are hydrogenated in the presence of 40 mg Raney nickel at ambient temperature and 3 bar hydrogen pressure. The catalyst is filtered off and the filtrate is freed from the solvent. White solid. Yield: 131 mg (trifluoroacetate); mass spectroscopy: [M+H]⁺=538.

The (R)- and (S)-enantiomers of this embodiment may be obtained by common methods known in the art. The (R)-enantiomer of this embodiment is of particular importance according to the invention.

### Example 12

### N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide

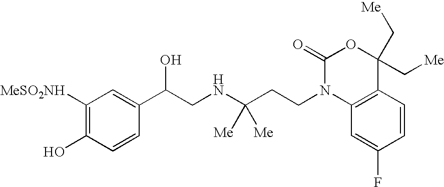

a) N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)-methanesulphonamide

246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-7-fluoro-1,4-dihydro-benzo[D][1,3]oxazin-2-one are and worked up analogously to Example 7a. However, the preparation of the reacted hydrochloride is omitted and the free base is purified by chromatography (reverse phase, acetonitrile/water gradient with 0.1% trifluoroacetic acid).

Yield: 220 mg (trifluoroacetate), HPLC: Rₜ=17.7 minutes (method A).

b) N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide

Prepared analogously to Example 11b from 210 mg N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)-methanesulphonamide trifluoroacetate. Grey solid.

Yield: 154 mg (trifluoroacetate); mass spectroscopy: [M+H]⁺=538.

The (R)- and (S)-enantiomers of this embodiment may be obtained by common methods known in the art. The (R)-enantiomer of this embodiment is of particular importance according to the invention.

### Example 13

### N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide

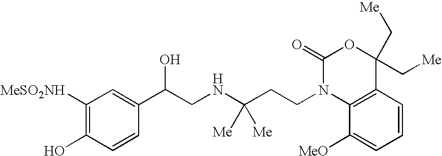

a) N-(2-benzyloxy-5-{2-[3-(4,4-diethyl-8-methoxy-2-oxo-4H-benzo[d][1,3]oxazin-1-yl)-1,1-dimethyl-propylamino]-1-hydroxy-ethyl}-phenyl)-methanesulphonamide

Reaction of 237 mg (0.625 mmol) N-[2-benzyloxy-5-(2-ethoxy-2-hydroxy-acetyl)-phenyl]-methanesulphonamide and 200 mg (0.624 mmol) 1-(3-amino-3-methyl-butyl)-4,4-diethyl-8-methoxy-1,4-dihydro-benzo[d][1,3]oxazin-2-one analogously to Example 7a. The crude product is dissolved in ethyl acetate and acidified to pH 2 with hydrochloric acid in ethyl acetate. The solvent is distilled off and the residue is stirred in diethyl ether. Then the hydrochloride thus obtained (330 mg) is further purified by chromatography. Yield: 90 mg (trifluoroacetate), HPLC: Rₜ=17.6 minutes (method A).

b) N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide

80 mg (0.118 mmol) N-(2-benzyloxy-5-{2-[3-(4,4-diethyl-8-methoxy-2-oxo-4H-benzo [d][1,3]oxazin-1-yl)-1,1-dimethyl-propylamino]-1-hydroxy-ethyl}-phenyl)-methanesulphonamide trifluoroacetate are hydrogenated analogously to Example 11b. Beige solid. Yield: 70 mg (trifluoroacetate); mass spectroscopy: [M+H]⁺=550.

The (R)- and (S)-enantiomers of this embodiment may be obtained by common methods known in the art. The (R)-enantiomer of this embodiment is of particular importance according to the invention.

### Example 14

### N-(5-{2-[3-(4,4-diethyl-6-methoxy-2-oxo-4H-benzo[d]][1,3oxazin-1-yl)-1,1-dimethyl-propylamino]-1-hydroxy-ethyl}-2-hydroxy-phenyl)-methanesulphonamide

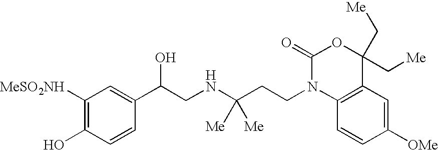

a) N-(2-benzyloxy-5-{2-[3-(4,4-diethyl-6-methoxy-2-oxo-4H-benzo[d][1,3]oxazin-1-yl)-1,1-dimethyl-propylamino]-1-hydroxy-ethyl}-phenyl)-methanesulphonamide

235 mg (0.619 mmol) N-[2-benzyloxy-5-(2-ethoxy-2-hydroxy-acetyl)-phenyl]-methanesulphonamide and 200 mg (0.624 mmol) 1-(3-amino-3-methyl-butyl)-4,4-diethyl-6-methoxy-1,4-dihydro-benzo[d][1,3]oxazin-2-one are reacted analogously to Example 7a. In contrast to that method the crude product is not precipitated as the hydrochloride, but purified by chromatography (reverse phase, acetonitrile/water gradient with 0.1% trifluoroacetic acid).

Yield: 150 mg (trifluoroacetate), HPLC: Rₜ=16.9 minutes (method A).

b) N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide

The target compound is prepared from N-(2-benzyloxy-5-{2-[3-(4,4-diethyl-6-methoxy-2-oxo-4H-benzo[d][1,3]oxazin-1-yl)-1,1-dimethyl-propylamino]-1-hydroxy-ethyl}-phenyl)-methanesulphonamide trifluoroacetate analogously to Example 11b. Grey solid (trifluoroacetate). Mass spectroscopy: [M+H]⁺=550.

The (R)- and (S)-enantiomers of this embodiment may be obtained by common methods known in the art. The (R)-enantiomer of this embodiment is of particular importance according to the invention.

Suitable preparations for administering the compounds of formula 1 include for example tablets, capsules, suppositories, solutions, powders, etc. The content of the pharmaceutically active compound(s) should be in the range from 0.05 to 90 wt.-%, preferably 0.1 to 50 wt.-% of the composition as a whole. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc, and/or agents for delayed release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations of active substances according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates or stabilisers such as alkali metal salts of ethylenediaminetetraacetic acid, optionally using emulsifiers and/or dispersants, while if water is used as diluent, for example, organic solvents may optionally be used as solubilisers or dissolving aids, and the solutions may be transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, preferably potato starch, gelatine and the like. Lubricants such as magnesium stearate, sodium laurylsulphate and talc may also be used to produce the tablets. In the case of aqueous suspensions the active substances may be combined with various flavour enhancers or colourings in addition to the abovementioned excipients.

When the compounds of formula 1 are used according to the invention for the treatment of the above-mentioned respiratory complaints it is particularly preferred to use preparations or pharmaceutical formulations which are suitable for inhalation. Inhalable preparations include inhalable powders, propellant-containing metered-dose aerosols or propellant-free inhalable solutions. Within the scope of the present invention, the term propellant-free inhalable solutions also includes concentrates or sterile ready-to-use inhalable solutions. The formulations which may be used within the scope of the present invention are described in more detail in the next part of the specification.

The inhalable powders which may be used according to the invention may contain 1 either on its own or in admixture with suitable physiologically acceptable excipients.

If the active substances 1 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred. Within the scope of the inhalable powders according to the invention the excipients have a maximum average particle size of up to 250 μm, preferably between 10 and 150 μm, most preferably between 15 and 80 μm. In some cases it may seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1, preferably with an average particle size of 0.5 to 10 μm, more preferably from 1 to 5 μm, is added to the excipient mixture. Processes for producing the inhalable powders according to the invention by grinding and micronising and lastly mixing the ingredients together are known from the prior art.

The inhalable powders according to the invention may be administered using inhalers known from the prior art.

The inhalation aerosols containing propellant gas according to the invention may contain the compounds 1 dissolved in the propellant gas or in dispersed form. The compounds 1 may be contained in separate formulations or in a common formulation, in which the compounds 1 are either both dissolved, both dispersed or in each case only one component is dissolved and the other is dispersed.

The propellant gases which may be used to prepare the inhalation aerosols are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The above-mentioned propellant gases may be used on their own or mixed together. Particularly preferred propellant gases are halogenated alkane derivatives selected from TG134a and TG227 and mixtures thereof.

The propellant-driven inhalation aerosols may also contain other ingredients such as co-solvents, stabilisers, surfactants, antioxidants, lubricants and pH adjusters. All these ingredients are known in the art.

The propellant-driven inhalation aerosols mentioned above may be administered using inhalers known in the art (MDIs=metered dose inhalers).

Moreover, the active substances 1 according to the invention may be administered in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is preferably up to 70 percent by volume, more particularly up to 60 percent by volume and most preferably up to 30 percent by volume. The remainder of the volume is made up of water. The solutions or suspensions containing 1 are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

If desired, the addition of editic acid (EDTA) or one of the known salts thereof, sodium edetate, as stabiliser or complexing agent may be omitted in these formulations. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as for example ascorbic acid, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in concentrations known from the prior art. The preservatives mentioned above are preferably present in concentrations of up to 50 mg/100 ml, more preferably between 5 and 20 mg/100 ml.

Preferred formulations contain, in addition to the solvent water and the active substance 1, only benzalkonium chloride and sodium edetate.

In another preferred embodiment, no sodium edetate is present.

The dosage of the compounds according to the invention is naturally highly dependent on the method of administration and the complaint which is being treated. When administered by inhalation the compounds of formula 1 are characterised by a high potency even at doses in the μg range. The compounds of formula 1 may also be used effectively above the μg range. The dosage may then be in the milligram range, for example.

In another aspect the present invention relates to the above-mentioned pharmaceutical formulations as such, which are characterised in that they contain a compound of formula 1, particularly preferably the above-mentioned pharmaceutical formulations administered by inhalation.

The following examples of formulations illustrate the present invention without restricting its scope:

### Examples of Pharmaceutical Formulations

| A) Tablets | per tablet | |
|---|---|---|
| active substance 1 | 100 | mg |
| lactose | 140 | mg |
| corn starch | 240 | mg |
| polyvinylpyrrolidone | 15 | mg |
| magnesium stearate | 5 | mg |
| | 500 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) Tablets | per tablet | |
|---|---|---|
| active substance 1 | 80 | mg |
| lactose | 55 | mg |
| corn starch | 190 | mg |
| microcrystalline cellulose | 35 | mg |
| polyvinylpyrrolidone | 15 | mg |
| sodium-carboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium carboxymethyl starch and the magnesium stearate are added and mixed in and the mixture is compressed to form tablets of a suitable size.

C) Ampoule solution

| | | |
|---|---|---|
| active substance 1 | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

D) Metering aerosol

| | |
|---|---|
| Active substance 1 | 0.005 |
| Sorbitan trioleate | 0.1 |
| Monofluorotrichloromethane and | ad 100 |
| TG134a:TG227 2:1 | |

The suspension is transferred into a conventional aerosol container with a metering valve. Preferably, 50 μl of suspension are delivered per spray. The active substance may also be metered in higher doses if desired (e.g. 0.02% by weight).

E) Solutions (in mg/100 ml)

| | | |
|---|---|---|
| Active substance 1 | 333.3 | mg |
| Benzalkonium chloride | 10.0 | mg |
| EDTA | 50.0 | mg |

| | |
|---|---|
| HCl (ln) | ad pH 3.4 |

This solution may be prepared in the usual manner.

F) Powder for inhalation

| | | |
|---|---|---|
| Active substance 1 | 12 | μg |
| Lactose monohydrate | ad 25 | mg |

The powder for inhalation is produced in the usual way by mixing the individual ingredients together.

We claim:

1. A method for treating a respiratory condition selected from the group consisting of obstructive pulmonary diseases, pulmonary emphysema, restrictive pulmonary diseases, interstitial pulmonary diseases, cystic fibrosis, bronchitis, bronchiectasis, ARDS (adult respiratory distress syndrome) and pulmonary oedema, comprising administering to a patient in need thereof a pharmaceutical composition comprising a compound of formula 1: 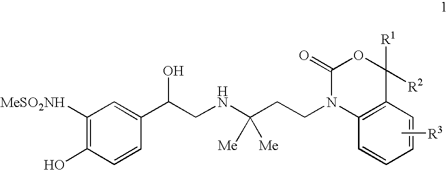
wherein
R¹ and R² are independently hydrogen, halogen or C₁-C₄-alkyl; or R¹ and R² together form a C₂-C₆-alkylene; and
R³ is hydrogen, halogen, OH, C₁-C₄-alkyl or —O—C₁-C₄-alkyl.

2. The method according to claim 1, wherein
R¹ and R² are independently hydrogen, fluorine, chlorine, methyl, ethyl, propyl or butyl; or R¹ and R² together form —CH₂—CH₂—, —CH₂—CH₂—CH₂—, —CH₂—CH₂—CH₂—CH₂— or —CH₂—CH₂—CH₂—CH₂—CH₂—; and
R³ is hydrogen, fluorine, chlorine, OH, methyl, ethyl, methoxy or ethoxy.

3. The method according to claim 1, wherein R¹ and R² are independently hydrogen, methyl, ethyl or propyl; or R¹ and R² together form —CH₂—CH₂—, —CH₂—CH₂—CH₂—, —CH₂—CH₂—CH₂—CH₂— or —CH₂—CH₂—CH₂—CH₂—CH₂—; and
R³ denotes hydrogen, fluorine, OH, methyl or methoxy.

4. The method according to claim 1, wherein the obstructive pulmonary disease is selected from the group consisting of: bronchial asthma, pediatric asthma, severe asthma, acute asthma, chronic bronchitis and chronic obstructive pulmonary disease (COPD).

5. The method according to claim 1, wherein the wherein the obstructive pulmonary disease is selected from the group consisting of bronchial asthma and chronic obstructive pulmonary disease (COPD).

6. The method according to claim 1, wherein the pulmonary emphysema has its origins in COPD or α1-proteinase inhibitor deficiency.

7. The method according to claim 1, wherein the restrictive pulmonary disease is selected from the group consisting of: allergic alveolitis, disease triggered by work-related noxious substances and disease caused by lung tumours.

8. The method according to claim 7, wherein the disease triggered by work-related noxious substance is asbestosis or silicosis.

9. The method according to claim 7, wherein the disease caused by lung tumors is lymphangiosis carcinomatosa, bronchoalveolar carcinoma or lymphomas.

10. The method according to claim 1, wherein the interstitial pulmonary disease is selected from the group consisting of: pneumonia, pneumonitis, collagenoses, granulomatoses, idiopathic interstitial pneumonia and idiopathic pulmonary fibrosis (IPF).

11. The method according to claim 10, wherein the pneumonia is caused by viral, bacterial, fungal, protozoal, helminthic or other pathogenic infection.

12. The method according to claim 10, wherein the pneumonitis is caused by aspiration and left heart insufficiency, radiation-induced pneumonitis or fibrosis.

13. The method according to claim 10, wherein the collagenoses is lupus erythematodes, systemic sclerodermy or sarcoidosis.

14. The method according to claim 10, wherein the granulomatoses is Boeck's disease.

15. The method according to claim 1, wherein the respiratory condition is selected from the group consisting of: cystic fibrosis or mucoviscidosis, bronchiectasis and ARDS (adult respiratory distress syndrome).

16. The method according to claim 1, wherein the bronchitis is caused by bacterial or viral infection, allergic bronchitis or toxic bronchitis.

17. The method according to claim 1, wherein the pulmonary oedema is a toxic pulmonary oedema caused by aspiration or inhalation of toxic and foreign substances.

18. The method according to claim 1, wherein the compound of formula 1 is an individual optical isomer, a mixture of individual enantiomers, one or more diastereomer or a racemate.

19. The method according to claim 18, wherein the compound is an enantiomerically-pure or diastereomerically-pure compound.

20. The method according to claim 1, wherein the compound of formula 1 is in the form of a free base or an acid addition salt prepared with a pharmacologically-acceptable acid.

21. The method according to claim 1, wherein the compound of formula 1 is in the form of a solvate, a hydrate or a solvate and hydrate.

22. A pharmaceutical composition comprising a compound of formula 1: 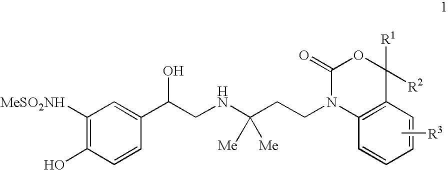
wherein
R¹ and R² are independently ethyl or propyl; or R¹ and R² together form —CH₂—CH₂—, —CH₂—CH₂—CH₂—, —CH₂—CH₂—CH₂—CH₂— or —CH₂—CH₂—CH₂—CH₂—CH₂—; and
R³ is hydrogen, fluorine, chlorine, OH, methyl, ethyl, methoxy or ethoxy.

23. The pharmaceutical composition according to claim 22, wherein R¹ and R² are identical.

24. The pharmaceutical composition according to claim 22, wherein R¹ and R² are ethyl or propyl; or R¹ and R² together form —CH₂—CH₂—, —CH₂—CH₂—CH₂—CH₂— or —CH₂—CH₂—CH₂—CH₂—CH₂—; and wherein
R³ is hydrogen, fluorine, OH, methyl or methoxy, preferably hydrogen.

25. The pharmaceutical composition according to claim 24, wherein R³ is hydrogen.

26. The pharmaceutical composition according to claim 22, wherein 1 is selected from the group consisting of:
N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide;
N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide;
N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide;
N-(5-{2-[3-(4,4-diethyl-2-oxo-4H-benzo [d][1,3]oxazin-1-yl)-1,1-dimethyl-propylamino]-1-hydroxy-ethyl}-2-hydroxy-phenyl)-methanesulphonamide;
N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide;
N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide;
N-(5-{2-[3-(4,4-diethyl-8-methoxy-2-oxo-4H-benzo[d][1,3]oxazin-1-yl)-1,1-dimethyl-propylamino]-1-hydroxy-ethyl} -2-hydroxy-phenyl)-methanesulphonamide; and
N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide.

27. The pharmaceutical composition according to claim 22, wherein the compound of formula 1 is an individual optical isomer, a mixture of individual enantiomers, one or more diastereomer or a racemate.

28. The pharmaceutical composition according to claim 22, wherein the compound of formula 1 is in the form of a free base or an acid addition salt prepared with a pharmacologically-acceptable acid.

29. The pharmaceutical composition according to claim 22, wherein the compound of formula 1 is in the form of a solvate, a hydrate or a solvate and hydrate.
